(19) United States
(12) Patent Application Publication
(10) Pub. No.: US 2008/0194548 A1
(43) Pub. Date: Aug. 14, 2008

(54) Drug Combination Therapy and Pharmaceutical Compositions for Treating Inflammatory Disorders

(76) Inventors: Michael J. Forrest, Shrewsbury, NJ (US); Julie A. DeMartino, Westfield, NJ (US); Michelle R. Flicker, Bridgewater, NJ (US); Augustin Melian, Woodcliff Lake, NJ (US); Samina Kanwar, Hoboken, NJ (US); Gary J. Romano, Wynnewood, PA (US)

Correspondence Address: MERCK AND CO., INC, P O BOX 2000, RAHWAY, NJ, 07065-0907, US

(21) Appl. No.: 11/794,857

(22) PCT Filed: Jan. 5, 2006
(86) PCT No.: PCT/US2006/000253

§ 371 Date: Feb. 19, 2008

Related U.S. Application Data
(60) Provisional application No. 60/641,707, filed on Jan. 6, 2005.

(51) Int. Cl. A61K 31/536; A61K 31/4375; A61K 31/351; A61K 31/40; A61P 11/00; A61P 9/10

(52) U.S. Cl. 514/230.5; 514/300; 514/460; 514/423

(57) ABSTRACT

A combination of a CCR2 antagonist and a statin is useful in the treatment and or prevention of inflammatory and other disorders, and methods of treating inflammatory and other disorders using a combination of a CCR2 antagonist and a statin.

### FIELD OF INVENTION

The present invention relates to novel methods and compositions for treating and preventing inflammatory disorders using combinations of CCR2 antagonists and statins.

### BACKGROUND OF THE INVENTION

It has been known for some time that chemokine receptors including CCR2 (and sub-types CCR2A and CCR2B) are important mediators of inflammatory and immunoregulatory disorders and diseases. A general review of the role of chemokines in disease is provided by Gerard and Rollins., Nature Immunol., 2(2),108-115 (2001).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis of monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)).

Thus, it is believed that MCP-1-induced CCR2 activation plays a major role in monocyte recruitment to inflammatory sites, and that antagonism of this activity produces a sufficient suppression of the immune response to produce therapeutic benefits in inflammatory diseases and conditions. Accordingly, agents which antagonize chemokine receptors such as the CCR2 receptor would be useful in treating such inflammatory diseases and conditions.

The statin family of drugs (HMG-CoA reductase inhibitors) is widely used in the management of cardiovascular disease, primarily by virtue of their documented ability to lower plasma levels of LDL cholesterol. Although lipid lowering is a major factor underlying the beneficial effects of statins in atherosclerosis and cardiovascular disease, it is evident that additional, immunomodulatory effects, may contribute to their efficacy. Accordingly, statins could be of benefit in a broad range of inflammatory conditions (for example, see Liao Int. J. Cardiol., 86, 5-18 (2000)). Consistent with this, Vollmer et al, Lancet, 363, 1607-1608 (2004), in an open label clinical study of 28 patients with definite relapsing-remitting multiple sclerosis, demonstrated a reduction of 44% in the number of gadolinium-enhancing lesions whilst on treatment with oral Simvastatin (80 mg). An earlier clue, to the non-lipid lowering benefit of statins, came from a trial of Pravastatin, demonstrating increased survival in cardiac transplant recipients independent of its cholesterol-lowering effect (Kobashigawa et al, New Engl. J. Med., 333, 621-627 (1995)). Subsequently, numerous effects of statins on aspects of inflammation and immune function have been reported. For example, Lovastatin has been shown to inhibit inducible nitric oxide synthetase (i-NOS) and the cytokine TNFα from microglia and astrocytes (Pahan et al, J. Clin. Invest., 100, 2671-2679 (1997)); Lovastatin and Simvastatin are reported to inhibit monocyte chemoattractant protein 1 (MCP1) production from either human peripheral blood mononuclear cells or from cultured endothelial cells. Moreover, statins are also reported to have anti-inflammatory effects in vivo including inhibition of the acute edematous response to carrageenan in mice (Sparrow et al, Arterioscler. Thromb. Vasc. Biol., 21, 115-121 (2000)) and inhibition of both developing or established collagen-induced arthritis in mice (Leung et al, J. Immunol., 170, 1524-1530 (2003)). The effects in mice are particularly important as statins do not lower LDL cholesterol levels in mice (Endo et al, Biochim. Biophys. Acta., 575, 266-276 (1979), Sparrow et al, Arterioscler. Thromb. Vasc. Biol., 21, 115-121 (2000)). Accordingly, any anti-inflammatory effects are independent of an effect on plasma LDL levels.

The LDL cholesterol lowering effect of statins is attributable to the ability of these compounds to inhibit the enzyme 3-hydroxy-3-methylglutaryl coenzyme A (HMG-CoA) reductase that converts acetoacetyl-CoA to mevalonate. Mevalonate is subsequently converted, via a multi-step pathway, to cholesterol. However, mevalonate is also a precursor for isoprenoids, including farnesylpyrophosphate (F—PP) and geranylgeranylpyrophosphate (G-PP). F—PP and G-PP play crucial roles in the post-translational modification of immunologically important proteins including, Rab, Rac, Rap, Rho and Ras. These proteins are crucial intracellular mediators of many leukocyte functions including maintenance of cell shape, cellular differentiation and proliferation, cell migration and secretory functions. Thus, blocking the production of isoprenoids with statins may modulate a broad range of cellular functions. The majority of the effects of statins on immunological function can be reversed by the addition of L-mevalonate, thus directly implicating a blockade of the mevalonate pathway. However, inhibition of the adhesion molecule, leukocyte function antigen 1 (LFA1), appears to be a direct effect of statins via binding to a regulatory site on the integrin molecule (Weitz-Schmidt et al, Nature Med., 7, 687-692 (2001)).

The precise mechanism(s) by which statins may exert anti-inflammatory and immunomodulatory effects is not known. It is evident that some of the effects of statins are dependent on inhibition of HMG-CoA reductase whereas other effects are independent of enzyme inhibition.

Nevertheless, sufficient evidence has been accumulated, both in vitro and in vivo, to support the concept for beneficial effects of statins in immune-based disorders that are not attributable to a lowering of LDL cholesterol.

### SUMMARY OF THE INVENTION

It has now been found that a wide range of inflammatory diseases and conditions can be more effectively treated, prevented and/or controlled by the co-administration of a CCR2 antagonist in combination with a statin than with either of these components alone. Thus, the present invention provides methods of treating, preventing and/or controlling inflammatory conditions in a mammalian patient in need thereof, which methods comprise administering to a patient an anti-inflammatory effective amount of a combination of a CCR2 antagonist and a statin. The invention also provides to pharmaceutical compositions comprising a CCR2 antagonist, a statin and a pharmaceutically acceptable carrier therefore. Additional objects of the invention will be evident from the following detailed description.

### DETAILED DESCRIPTION

The present invention provides methods of treating, preventing and/or reducing the risk of onset of inflammatory disorders by administration to a patient in need thereof a combination of a CCR2 antagonist and a statin, in amounts sufficient to result in an anti-inflammatory effect, either by reducing inflammation or by preventing inflammation.

In the novel method of treatment described herein, the two active ingredients can be administered combined in a single dosage form or as two separate dosage forms, each containing one of the active ingredients. Thus, the instant pharmaceutical combination comprising a CCR2 antagonist in combination with a statin inhibitor includes administration of a single pharmaceutical dosage formulation which contains both the CCR2 antagonist and the statin, as well as administration of each active agent in its own separate pharmaceutical dosage formulation. Where separate dosage formulations are used, the CCR2 antagonist and the statin can be administered at essentially the same time, i.e., concurrently, or at separately staggered times, i.e., sequentially. The instant pharmaceutical combination is understood to include all such medically acceptable dosing regimens. Administration in these various ways are suitable for the present invention as long as the beneficial pharmaceutical effect of the CCR2 antagonist and the statin are realized by the patient at substantially the same time. Such beneficial effect is preferably achieved when the target blood level concentrations of each active drug are maintained at substantially the same time. It is preferred that the CCR2 antagonist and the statin be co-administered concurrently on a once-a-day dosing schedule; however, varying dosing schedules, such as the CCR2 antagonist once, twice or more times per day and the statin once, twice or more times per day, is also encompassed herein. A single oral dosage formulation comprised of both a CCR2 antagonist and the statin is preferred. Additional embodiments of the invention include pharmaceutical compositions comprising a combination of a CCR2 antagonist and a statin plus one or more pharmaceutically acceptable carriers.

The present invention is not limited to combinations comprising only particular CCR2 antagonists. Thus, examples of CCR2 antagonists that may be used in connection with the present invention include, but are not limited to, compounds such as:
N-((1R,3S)-3-isopropyl-3-{[3-(trifluoromethyl)-7,8-dihydro-1,6-naphthyridin-6(5H)-yl]carbonyl}cyclopentyl)-N-[(3S,4S)-3-methoxytetrahydro-2H-pyran-4-yl]amine:

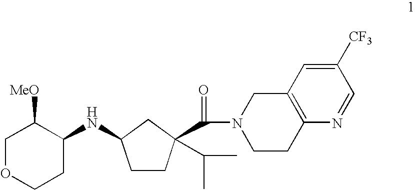

(hereafter, “Compound 1”) and pharmaceutically acceptable salts thereof;
3 [(3S,4R)-1-((1R,3S)-3-isopropyl-2-oxo-3-{[6-(trifluoromethyl)-2H-1,3-benzoxazin-3(4H)-yl]methyl}cyclopentyl)-3-methylpiperidin-4-yl]benzoic acid:

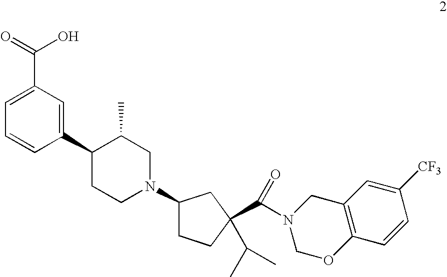

(hereafter, “Compound 2”) and pharmaceutically acceptable salts thereof;
(3S,48)-N-((1R,3S)-3-isopropyl-3-{[7-(trifluoromethyl)-3,4-dihydroisoquinolin-2(1B)-yl]carbonyl}cyclopentyl)-3-methyltetrahydro-2H-pyran-4-aminium:

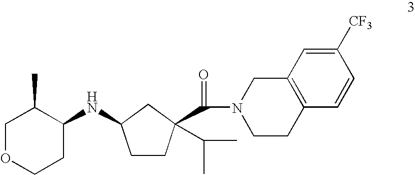

(hereafter, “Compound 3”) and pharmaceutically acceptable salts thereof;
3-[(3S,4R or 3R,4S)-1-((1R,3S)-3-Isopropyl-3-{[6-(trifluoromethyl)-2H-1,3-benzoxazin-3(4H)-yl]carbonyl}cyclopentyl)-3-methylpiperidin-4-yl]benzoic acid:

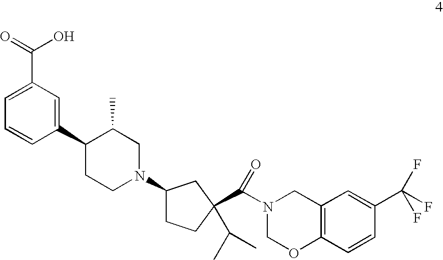

(hereafter, “Compound 4”) and pharmaceutically acceptable salts thereof;
INCB3284; Eotaxin-3; compounds such as those described in WO04/050024, and other CCR2 antagonists described in the scientific and patent literature.

Examples of statins that may be used with the present invention include but are not limited to the lactonized and dihydroxy open acid forms and pharmaceutically acceptable salts and esters thereof of: lovastatin (MEVACOR®, see U.S. Pat. No. 4,342,767); simvastatin (ZOCOR®; see U.S. Pat. No. 4,444,784); pravastatin, particularly the sodium salt thereof (PRAVACHOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); rosuvastatin, particularly the calcium salt thereof (CRESTOR®; see U.S. Pat. No. Re 37314); and pitavastatin also referred to as NK-104 (see PCT international publication number WO 97/23200). The structural formulas of several of these statins and additional HMG-CoA reductase inhibitors are described at page 87 of M. Yalpani, “Cholesterol Lowering Drugs”, Chemistry & Industry, pp. 85-89 (5 Feb. 1996). The preferred statin for use in this invention is simvastatin.

Herein, the term “pharmaceutically acceptable salts” shall mean nontoxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)-aminomethane.

The term “patient” is intended herein to mean humans or animals who take a statin in combination with a CCR2 antagonist for any of the uses described herein. Administering of the drug combination to the patient includes both self-administration and administration to the patient by another.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

The compounds of use in this invention may have one or more chiral centers and the present compounds may occur as racemates, racemic mixtures and as individual diastereomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

In one aspect of the invention, a method of treating or preventing inflammatory disorders is disclosed in a mammalian patient in need of such treatment, which comprises administering to the patient a CCR2 antagonist and a statin, or salts or hydrates thereof, in amounts that are effective for treating or preventing inflammation.

The invention includes the method wherein the statin and CCR2 antagonist are administered combined in a single dosage form, as well as the method wherein the statin and CCR2 antagonist are administered as separate dosage forms substantially concurrently.

The invention also includes a pharmaceutical composition which is comprised of a statin and CCR2 antagonist, or salts or hydrates thereof, in combination with a pharmaceutically acceptable carrier.

More particularly, the composition is described wherein the statin is selected from lovastatin, simvastatin, pravastatin, fluvastatin, atorvastatin and rosuvastatin, and the CCR2 inhibitor is selected from Compound 1, Compound 4, and INCB3284. Even more particularly, the composition is described wherein the statin is simvastatin or a salt thereof, and the CCR2 antagonist is selected from Compound 1 and Compound 4.

The dosage regimen utilizing a statin in combination with a CCR2 antagonist is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

An effective amount of the combination is that amount that will relieve the subject being treated of the symptoms of the particular condition, or prevent such symptoms, and the specific dose level and frequency of dosage may vary and will depend upon a variety of factors including the activity of the specific compounds used in combination, the metabolic stability and length of action of the compounds, the age, body weight, general health, sex, diet, mode and time of administration, rate of excretion, the severity of the particular condition and the host undergoing therapy. However, dosage levels of the CCR2 antagonist on the order of about 0.001 mg/kg to about 250 mg/kg of body weight per day, typically about 0.005 to about 100 mg/kg, more particularly about 0.01 to about 50 mg/kg and especially about 0.05 to about 10 mg/kg per day are useful in the novel method of treatment. Dosage levels of the statin of about 0.1 to 500 mg/kg of body weight per day, typically about 0.5 to about 250 mg/kg, more particularly about 5 to about 100 mg/kg and especially about 5 to about 50 mg/kg of body weight per day are useful in the novel method of this invention. In a preferred combination, a composition is described wherein simvastatin or a salt thereof is present in an amount ranging from about 1 to about 10 mg, and one of Compounds 1, 2, 3 or 4 or a salt thereof, is present in an amount ranging from about 0.5 mg to about 20 mg, more preferably about 1.0 mg to about 10 mg.

For the treatment of inflammatory and other conditions, the active ingredients, separately or in combination, may be administered orally, topically, parenterally, by inhalation, spray, rectally or intravaginally in formulations containing pharmaceutically acceptable carriers.

The term parenteral as used herein includes subcutaneous injections, intravenous, intramuscular, intracisternal injection or infusion techniques.

Additional active agents may be used in combination with the statin and CCR2 antagonist in a single dosage formulation, or may be administered to the patient in a separate dosage formulation, which allows for concurrent or sequential administration. One or more additional active agents may be administered with the instant combination therapy. Diseases or conditions of humans or other species which can be treated with the combinations of the present invention include, but are not limited to:

inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; and cancers, including cancers with leukocyte infiltration of the skin or organs and other cancers. The inventive combinations of the invention likewise may also be useful in the treatment and prevention of stroke, neurodegenerative conditions including but not limited to Alzheimer's disease, amyotrophic lateral sclerosis (ALS) and Parkinson's disease, obesity, type II diabetes, neuropathic and inflammatory pain, and Guillain Barre syndrome. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis, fibrosis, and chronic obstructive pulmonary disease.

Additional diseases or conditions of humans or other species, which can be treated with the combinations of the instant invention include or involve but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infectious diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum).

Although the combinations of the present invention are further useful in treating, preventing, ameliorating, controlling or reducing the risk of a wide variety of inflammatory and immunoregulatory disorders and diseases, allergic conditions, atopic conditions, as well as autoimmune pathologies, in a specific embodiment the present invention is directed to the use of the subject compounds for treating, preventing, ameliorating, controlling or reducing the risk of autoimmune diseases such as rheumatoid arthritis, psoriatic arthritis and multiple sclerosis.

The separate active agents or the novel composition of this invention may be in a form suitable for oral use, for example, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions and typically such compositions contain one or more agents selected from the group consisting of sweetening agents, flavouring agents, colouring agents and preservatives in order to provide pharmaceutically elegant and palatable preparations. These excipients may be for example, diluents such as lactose, calcium carbonate, sodium carbonate, calcium phosphate or sodium phosphate; granulating and disintegrating agents, for example, corn starch or alginic acid; binding agents, for example starch, gelatin or acacia, and lubricating agents, for example, magnesium stearate, stearic acid or talc.

The tablets may be uncoated or they may be coated. Coating can be included to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pats. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, tragacanth and acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The individual agents or the pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy-ethylene sorbitan monooleate. The emulsions may also contain sweetening and flavouring agents. Syrups and elixirs may be formulated with sweetening agents, for example glycerol, propylene glycol, sorbitol or sucrose. Such formulations may also contain demulcents, preservatives, flavourants and colouring agents.

The pharmaceutical compositions may be in the form of a sterile injectable aqueous or oleagenous suspension. This suspension may be formulated according to the known art using those suitable dispersing or wetting agents and suspending agents which have been mentioned above.

Injectable compositions are typically in the form of sterile solutions or suspensions, which include the active ingredient in a parenterally-acceptable diluent. Among these are sterile water, dextrose 5% in water (D5W), Ringer's solution and isotonic saline, as well as mixtures thereof. Cosolvents such as ethanol, propylene glycol or polyethylene glycols may also be used. Sterile, injectable oil is occasionally employed as a solvent or suspending medium in intramuscular preparations. A representative example is peanut oil. In addition, fatty acids such as oleic acid, preservatives, buffers and local anesthetics find use in the preparation of intramuscular injectables.

The combination of active ingredients may also be administered rectally or intravaginally as suppositories. These can be prepared by mixing the drug with a suitable non-irritating excipient which is solid at ordinary room temperature but molten at normal or elevated body temperature. Examples of such materials include cocoa butter and polyethylene glycols.

For topical use, creams, ointments, gels, solutions, suspensions and the like containing the compound are employed. (For purposes of this application, topical application includes mouth washes and gargles, as well as transdermal applications.) Topical formulations are comprised of a pharmaceutical carrier, which may include, e.g., cosolvents, emulsifiers, penetration enhancers, preservatives or emollients.

The active ingredients are combined with the carrier to produce the dosage form. For example, a formulation intended for oral administration may contain from as low as about 0.1 mg of the novel combination to as high as about 5 g of combination per dose, compounded with an appropriate and convenient amount of carrier material which may vary from about 5 to about 95 percent of the total composition.

As used herein, the term “treating” or “treat” or “treatment” encompasses not only treating a patient to relieve the sis and symptoms of the disease or condition, but also prophylactically treating an asymptomatic patient to prevent the onset or progression of the disease or condition.

The following Examples 1 and 2 are illustrative of results expected when co-administering the inventive combinations of CCR2 antagonist and statin.

### EXAMPLE 1

### Test Methods

Female C57BL/6 mice are injected subcutaneously with 100 μg of myelin oligodendrocyte glycoprotein peptide 35-55 (MOGp35-55) emulsified in complete Freund's adjuvant containing Mycobacterium tuberculosis (4 mg/kg: strain H37Ra). At the time of immunization and 2 days later mice also receive pertussis toxin (150 ng) administered intravenously in a tail vein. The initiation and progression of disease activity is assessed on a daily basis using a “disease activity” score. The disease activity score evaluates individual animals with respect to their physical behavior. Thus, 0=no change; 1=limp tail; 2=altered gait; 3=hind limb paralysis; 4=fore limb paralysis; 5=dead or moribund.

The effects of pharmacological agents in this model are evaluated by administering test compositions in a volume of 10 mL/kg by oral gavage immediately prior to the subcutaneous injection of MOGp35-55 and daily thereafter for the duration of study. Test compositions include a vehicle (0.5% methylcellulose), low and high doses of Compounds 1, 2 and 4, simvastatin, atorvastatin, Compound I co-dosed with simvastatin, Compound 2 co-dosed with simvastatin, Compound 4 co-dosed with simvastatin, Compound 1 co-dosed with atorvastatin, Compound 2 co-dosed with atorvastatin and Compound 4 co-dosed with atorvastatin.

### EXAMPLE 2

### Results

In mice that receive the CCR2 receptor antagonists Compound 1, Compound 2 or Compound 4, the onset of disease symptoms is delayed in a dose-dependent manner compared to animals that receive vehicle. The maximal extent and duration of disease activity is also reduced compared with control animals. Similarly, in mice that receive statin simvastatin or atorvastatin the onset of disease symptoms is delayed in a dose-dependent manner compared to animals that receive vehicle. As with the CCR2 receptor antagonists, the maximal extent and duration of disease activity is reduced compared with control animals.

However, in mice that are co-dosed with a CCR2 receptor antagonists and a statin, the onset of disease symptoms is delayed to a greater extent than the sum of the delays attributable to either the CCR2 antagonist or the statin individually. Similarly, in mice that are co-dosed with a CCR2 receptor antagonists and a statin, the maximal extent and duration of disease activity is reduced to a greater extent than the sum of the delays attributable to either the CCR2 antagonist or the statin individually.

### EXAMPLES 3 AND 4

### Tablet Preparation

Tablets containing 5 mg and 10 mg of simvastatin and 5 mg of Compound 1 were prepared as follows:

| | Example 3 | Example 4 |
|---|---|---|
| Simvastatin | 5.0 mg | 10.0 mg |
| Compound 1 | 5.0 mg | 5.0 mg |
| Microcrystalline cellulose | 42.0 mg | 39.5 mg |
| Modified food starch | 42.0 mg | 39.5 mg |
| Magnesium stearate | 1.0 mg | 1.0 mg |

All of the active ingredients, cellulose, and a portion of the corn starch are mixed and granulated to 10% corn starch paste. The resulting granulation is sieved, dried and blended with the remainder of the corn starch and magnesium stearate. The resulting granulation is then compressed into tablets.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds and compositions of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method of treating or preventing inflammatory disorders in a mammalian patient in need of such treatment or prevention, said method comprising administering to the patient a CCR2 antagonist or a salt or hydrate thereof, and a statin or a salt or hydrate thereof, in amounts that are effective for treating or preventing inflammation.

2. A method of treating or preventing inflammatory disorders in a mammalian patient in need of such treatment or prevention, said method comprising administering to the patient a CCR2 antagonist or a salt or hydrate thereof, and a statin or a salt or hydrate thereof, in amounts that are effective for treating or preventing inflammation,
wherein said CCR2 antagonist is selected from N-((1R,3S)-3-isopropyl-3-{[3-(trifluoromethyl)-7,8-dihydro-1,6-naphthyridin-6(5H)-yl]carbonyl}cyclopentyl)-N-[(3S,4S)-3-methoxytetrahydro-2H-pyran-4-yl]amine, 3 [(3S,4R)-1-((1R,3S)-3-isopropyl-2-oxo-3-{[6-(trifluoromethyl)-2H-1,3-benzoxazin-3(4H)-yl]methyl}cyclopentyl)-3-methylpiperidin-4-yl]benzoic acid, (3S,4S)—N-((1R,38)-3-isopropyl-3-{[7-(trifluoromethyl)-3,4-dihydroisoquinolin-2(1H)-yl]carbonyl}cyclopentyl)-3-methyltetrahydro-2H-pyran-4-aminium, 3-[(3S,4R or 3R,4S)-1-((1R,3S)-3-Isopropyl-3-{[6-(trifluoromethyl)-2H-1,3-benzoxazin-3(4H)-yl]carbonyl}cyclopentyl)-3-methylpiperidin-4-yl]benzoic acid, and INCB33284, Eotaxin-3, and salts and hydrates thereof, and
wherein said statin is selected from lovastatin, simvastatin, pravastatin, fluvastatin, atorvastatin, rosuvastatin, and pitavastatin, and salts and hydrates thereof.

3. The method according to claim 2, wherein said CCR2 antagonist is selected from N-((1R,3S)-3-isopropyl-3-{[3-(trifluoromethyl)-7,8-dihydro-1,6-naphthyridin-6(5H)-yl]carbonyl}cyclopentyl)-N-[(3S,4S)-3-methoxytetrahydro-2H-pyran-4-yl]amine, 3 [(3S,4R)-1-((1R,3S)-3-isopropyl-2-oxo-3-{[6-(trifluoromethyl)-2H-1,3-benzoxazin-3(4H)-yl]methyl}cyclopentyl)-3-methylpiperidin-4-yl]benzoic acid, (3S,4S)—N-((1R,3S)-3-isopropyl-3-{[7-(trifluoromethyl)-3,4-dihydroisoquinolin-2(1H)-yl]carbonyl}cyclopentyl)-3-methyltetrahydro-2H-pyran-4-aminium, 3-[(3S,4R or 3R,4S)-1-((1R,3S)-3-Isopropyl-3-{[6-(trifluoromethyl)-2H-1,3-benzoxazin-3(4H)-yl]carbonyl}cyclopentyl)-3-methylpiperidin-4-yl]benzoic acid, and salts and hydrates thereof, and said statin is simvastatin.

4. The method according to claim 2, wherein said CCR2 antagonist is INCB3284.

5. The method of claim 2, wherein said statin is selected from lovastatin, simvastatin, pravastatin, fluvastatin, atorvastatin and rosuvastatin.

6. The method of claim 2, wherein said CCR2 antagonist and said statin are administered concurrently as separate dosage forms.

7. The method of claim 2, wherein said CCR2 antagonist and said statin comprise a single dosage form.

8. The method of claim 2, wherein said disorder is selected from multiple sclerosis and rheumatoid arthritis.

9. The method of claim 2, wherein said disorder is chronic obstructive pulmonary disease.

10. The method of claim 2, wherein said inflammatory disorder is atherosclerosis.

11. A pharmaceutical composition which comprises a CCR2 antagonist, a statin and an inert carrier,
wherein said CCR2 antagonist is selected from N-((1R,3S)-3-isopropyl-3-{[3-(trifluoromethyl)-7,8-dihydro-1,6-naphthyridin-6(5H)-yl]carbonyl}cyclopentyl)-N-[(3S,4S)-3-methoxytetrahydro-2H-pyran-4-yl]amine, 3 [(3S,4R)-1-((1R,3S)-3-isopropyl-2-oxo-3-{[6-(trifluoromethyl)-2H-1,3-benzoxazin-3(4H)-yl]methyl}cyclopentyl)-3-methylpiperidin-4-yl]benzoic acid, (3S,48)-N-((1R,3S)-3-isopropyl-3-{[7-(trifluoromethyl)-3,4-dihydroisoquinolin-2(11)-yl]carbonyl}cyclopentyl)-3-methyltetrahydro-2H-pyran-4-aminium, 3-[(3S,4R or 3R,4S)-1-((1R,3S)-3-Isopropyl-3-{[6-(trifluoromethyl)-2H-1,3-benzoxazin-3(4H)-yl]carbonyl}cyclopentyl)-3-methylpiperidin-4-yl]benzoic acid, and INCB3284, Eotaxin-3, and salts and hydrates thereof, and
wherein said statin is selected from lovastatin, simvastatin, pravastatin, fluvastatin, atorvastatin, rosuvastatin, and pitavastatin, and salts and hydrates thereof.

12. The composition of claim 10, wherein said CCR2 antagonist is selected from N-((1R,3S)-3-isopropyl-3-{[3-(trifluoromethyl)-7,8-dihydro-1,6-naphthyridin-6(5H)-yl]carbonyl}cyclopentyl)-N-[(3S,4S)-3-methoxytetrahydro-2H-pyran-4-yl]amine, 3 [(3S,4R)-1-((1R,3S)-3-isopropyl-2-oxo-3-{[6-(trifluoromethyl)-2H-1,3-benzoxazin-3(4H)-yl]methyl}cyclopentyl)-3-methylpiperidin-4-yl]benzoic acid, (3S,4S)—N-((1R,3S)-3-isopropyl-3-{[7-(trifluoromethyl)-3,4-dihydroisoquinolin-2(1H)-yl]carbonyl}cyclopentyl)-3-methyltetrahydro-2H-pyran-4-aminium, 3-[(3S,4R or 3R,4S)-1-((1R,3S)-3-Isopropyl-3-{[6-(trifluoromethyl)-2H-1,3-benzoxazin-3(4H)-yl]carbonyl}cyclopentyl)-3-methylpiperidin-4-yl]benzoic acid, and salts and hydrates thereof, and said statin is simvastatin.

13. The composition of claim 10, wherein said CCR2 antagonist is INCB3284.

14. The composition of claim 10, wherein said CCR2 antagonist and said statin are administered concurrently as separate dosage forms.

15. The composition of claim 10, wherein said CCR2 antagonist and said statin comprise a single dosage form.
